(19) United States
(12) Patent Application Publication
(10) Pub. No.: US 2026/0315336 A1
(43) Pub. Date: Oct. 15, 2026

(54) NAIL REPAIR PATCH

(71) Applicant: Keejung BARNG, Pyeongtaek-si, Gyeonggi-do (KR)

(72) Inventor: Keejung BARNG, Pyeongtaek-si, Gyeonggi-do (KR)

(21) Appl. No.: 18/869,691

(22) PCT Filed: Oct. 15, 2024
(86) PCT No.: PCT/KR2024/015609

§ 371 Date: Nov. 26, 2024

(30) Foreign Application Priority Data
Nov. 27, 2023 (KR) 10-2023-0166714

(51) Int. Cl. A61K 8/02; A61K 8/26; A61K 8/65; A61K 8/73; A61Q 3/02; A61Q 17/00

(52) U.S. Cl. CPC A61K 8/0208; A61K 8/26; A61K 8/65; A61K 8/732; A61Q 3/02; A61Q 17/005; A61K 2800/74

(57) ABSTRACT

The present invention relates to a multi-functional nail repair patch for both of an antibacterial function and a beauty function. The repair patch according to the present invention satisfies a consumer's needs for antibacterial and beauty functions, improves conveniences of use through which it can be attached easily to the nail only with the use of a small amount of water, is replaced with new one after one to two days to provide an excellent sanitary state, and gives a fun to the user because the patch having a first pattern is changed to the patch having a second pattern, so that it can be applicable in industrial fields and optimized in the commercial value thereof.

### TECHNICAL FIELD

The present invention relates to a multi-functional nail repair patch having an antibacterial function and a beauty function.

### BACKGROUND ART

Reasons for color or shape change in nails are as follows:
About 50% of color or shape change in nails are caused by fungal infection. Further, a change in color or shape of the nails occurs due to injury, inherent deformation, psoriasis, lichen planus, infection, etc. The fungal infection is caused by Trichophyton rubrum, Trichophyton mentagrophytes, etc. Referring to prior art document 1, there is disclosed a pharmaceutical composition for preventing or treating onychomycosis that comprises urea, fumaric acid, 1,3-butylene glycol, gel-forming copolymer, a crosslinking agent, and water (See claim 1).

A nail care market trend is described below:
It is predicted that a global nail care market has an annual growth rate of 8.0% during a period from 2022 to 2031. It is also predicted that in 2031, the global nail care market will expand to a scale of 23. 1 billion dollars. Various products such as liquid manicures, gel nails that are cured through a UV cure machine, dry type stickers, and the like have been shipped under a consumer's needs. Referring to prior art document 2, there is disclosed a nail sticker and a method for manufacturing the same, and the nail sticker includes a PET film, an acrylic adhesion layer, a vacuum deposition coating layer, a color layer, a glitter layer, and a protective tape (See claim 1). Referring further to prior art document 3, there is disclosed a semi-cured gel nail sticker including a release film layer, a top gel layer having a UV-curing resin, a print layer, a color layer, a curable adhesive layer, release paper, and a UV light-shielding film (See claim 1).

Consumer needs for multiple functions are explained below:
A consumer having the nails changing in color or shape has great needs for a product having both a repair function and a beauty function. Such a repair function exists, but if the product has a general band shape or transparent color, the beauty function becomes weak to cause the consumer's desire for beauty to increase. In a state where his or her nails do not change in color or shape, however, even in the case of the consumer who needs protective care because his or her nails have subtle injuries or are seriously dried due to existing nail care, he or she has such a desire for beauty. Referring further to prior art document 4, there is disclosed a gel nail sticker including a release film layer, a top gel layer having a UV-curing resin, and an adhesive layer having an antibacterial agent, and the antibacterial agent has copper-boehmite composite particles (See claim 1).

Consumer needs for a product easily attached and detached to and from a nail are explained below:
Among consumers, some consumers do not like the nail care products that are hard to be attached to their nails or cause subtle injuries on their nails due to attachment to the nails for a long period of time. Therefore, there is a need to develop a product that is capable of being easily attached and detached to and from a nail.

### DISCLOSURE OF THE INVENTION

### Technical Problems

It is a first object of the present invention to provide a multi-functional nail repair patch having a repair function and a beauty function. It is a second object of the present o provide a nail repair patch having an antibacterial function against fungus, a moisturizing function, and a beauty function. It is a third object of the present invention to provide a nail repair patch easily attachable and detachable to and from a nail.

### Technical Solutions

A solution 1 provides a nail repair patch including:
a water adhesion layer 100 for providing an attachable function to a nail by means of water;
a functional layer 200 having an antibacterial constituent and a nutrition constituent; and
a beauty cover layer 300 having a given pattern for a sense of beauty printed on a top thereof and an adhesion constituent applied to an underside thereof,
wherein the nail repair patch has
the water adhesion layer 100 comprising:
a water adhesion constituent 110 for providing a water-soluble adhesion function; and
a porous support layer 120 for supporting the water adhesion constituent 110.

According to the solution 1, a solution 2 provides the nail repair patch wherein
the water adhesion constituent 110 includes starch or gelatin.

According to the solution 1 or 2, a solution 3 provides
the nail repair patch wherein
the functional layer 200 includes
the antibacterial constituent 210 and the nutrition constituent 230 formed
on a top or underside of a porous support layer 220, or
on the underside of the porous support layer 220.

According to the solution 3, a solution 4 provides the nail repair patch wherein
Each of the antibacterial constituent 210 and the nutrition constituent 230 is made in the form of a single liquid.

According to the solution 1 or 2, a solution 5 provides the nail repair patch wherein
the antibacterial constituent 210 includes a constituent having an antibacterial function against fungus.

According to the solution 1 or 2, a solution 6 provides the nail repair patch wherein
the nutrition constituent 230 includes a constituent for the moisturization and nutrition of the nail.

According to the solution 1, a solution 7 provides the nail repair patch wherein
the adhesion constituent on the underside of the beauty cover layer 300 includes
the same constituent as the water adhesion constituent 110.

According to the solution 3, a solution 8 provides the nail repair patch wherein
the porous support layer 220 has smaller ventilation holes than the porous support layer 120.

According to the solution 1, a solution 9 provides the nail repair patch wherein
the water adhesion constituent 110 is formed by a process in which
a solution containing a water adhesion material is sprayed and dried, by a process in which after the solution has been sprayed, the powder of the solution is applied and dried, or by a process in which the water adhesion material is made into a hydrogel and the hydrogel is applied.

According to the solution 1, a solution 10 provides the nail repair patch wherein
the antibacterial constituent 210 and the nutrition constituent 230 are formed by a process in which
an antibacterial material solution or a nutrition material solution is sprayed and dried, by a process in which after the solution has been sprayed, the powder of the solution is applied and dried, or by a process in which an antibacterial material or a nutrition material is made into a hydrogel and the hydrogel is applied.

### Advantageous Effects of the Invention

According to a first effect of the present invention, the nail repair patch satisfies a consumer's needs for antibacterial and beauty functions. According to a second effect of the present invention, the nail repair patch improves a user's conveniences through which it can be easily attached to and detached from the nail with the use of a given amount of water. According to a third effect of the present invention, the nail repair patch is replaced with new one after one to two days to improve sanitation thereof. According to a fourth effect of the present invention, the nail repair patch gives a fun to the user because the patch having a first pattern changes to the patch having a second pattern.

### BRIEF DESCRIPTION OF DRAWINGS

FIG. 1 is a sectional view showing a nail repair patch according to a first embodiment of the present invention.

FIG. 2 is a sectional view showing a nail repair patch according to a second embodiment of the present invention.

FIG. 3 is a sectional view showing a nail repair patch according to a third embodiment of the present invention.

### MODE FOR INVENTION

The terms used in the present invention are to be given their ordinary and customary meaning to a person of ordinary skill in the art, and are not to be limited to a special or customized meaning unless expressly so defined herein. It should be noted that the use of particular terminology when describing certain features or aspect of the disclosure should not be taken to imply that the terminology is being re-defined herein to be restricted to include any specific characteristics of the features or aspects of the disclosure with which that terminology is associated.

Before the present invention is disclosed and described, it is not limited to suggested manufacturing examples, experimental examples, or embodiments, and therefore, it is to be understood that the disclosed embodiments are merely exemplary of the invention, which can be embodied in various forms. Those skilled in the art will envision many other possible variations within the scope of the present invention.

The term ‘repair’ used in the specification refers to prevention of color or shape change of nails or improvement therein, which includes an antibacterial function and/or moisturizing function. Further, the term ‘patch’ represents a term made by combining a ‘piece’ based on the dictionary and a repair function within the entire technical scope of the present invention.

### Embodiment 1. Multi-Functional Nail Repair Patch for an Antibacterial Function and a Beauty Function

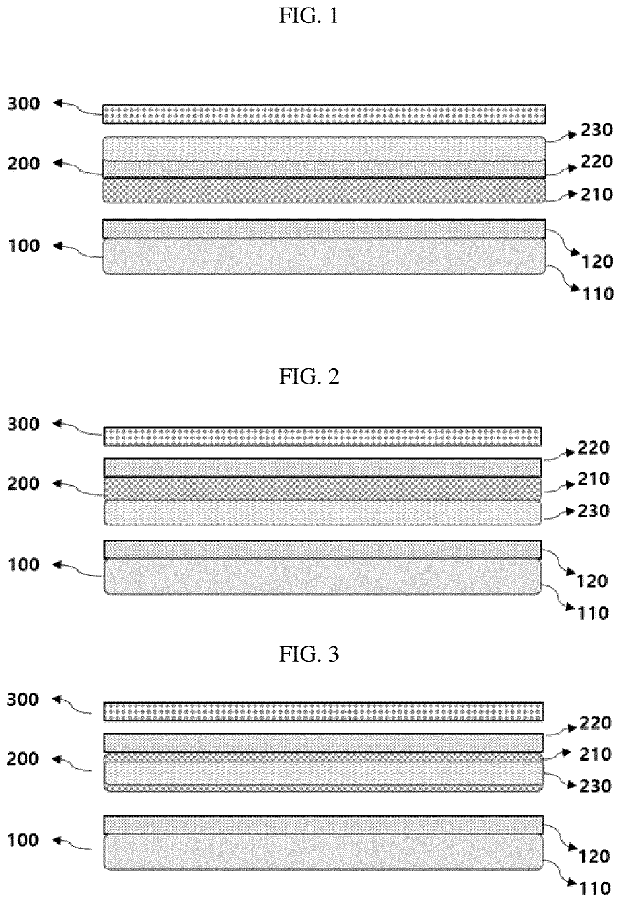

A nail repair patch according to a first embodiment of the present invention includes a water adhesion layer 100, a functional layer 200, and a beauty cover layer 300 (See FIG. 1).

The water adhesion layer 100 is attachable to a nail through saliva or a small amount of water and includes a water adhesion constituent 110 and a porous support layer 120.

The water adhesion constituent 110 has a water-soluble adhesion function. That is, the water adhesion constituent 110 provides an adhesion function only with a small amount of water. For example, the water adhesion constituent 110 includes starch and/or gelatin. According to design conditions, the water adhesion constituent 110 may include protein, glycolipid, etc. that have the same function as above.

The porous support layer 120 serves to support the water adhesion constituent 110, while having the shape of a patch. For example, the porous support layer 120 is made of porous paper and/or porous non-woven fabric and have the shape of a nail to cover the entire surface of the nail whose color or shape is changed. According to design conditions, the porous support layer 120 may be made of a material that is porous, flexible, and coated with a solution or hydrogel.

Referring to a method for manufacturing the water adhesion layer 100, first, a solution that is made by dissolving starch in water and/or a solution that is made by dissolving collagen in water are/is sprayed into the porous support layer 120 and then dried. Desirably, the water adhesion layer 100 is made to have a function of being kept attached to the nail for one to two days. That is, the process of spraying and drying the solution having the adhesion constituent is carried out at least two or more times, so that the water adhesion constituent 110 is formed to have a thickness greater than or equal to the porous support layer 120.

Otherwise, powder that is made by drying and powderizing the solution made by dissolving starch in water and/or powder that is made by drying and powderizing the solution made by dissolving collagen in water are/is used, together with the above-mentioned solution. In this case, the process of spraying the solution having the adhesion constituent, applying the powder, and drying the applied powder is carried out at least two or more times. In this case, even though the water adhesion constituent 110 is formed to have a thickness less than the porous support layer 120, it can keep its adhesion force.

Otherwise, the water adhesion constituent 110 is made of a hydrogel and thus applied to the porous support layer 120. That is, the water adhesion constituent 110 is made of a hydrogel consisting of starch and/or collagen, a gelling agent, a crosslinking agent, and water and thus applied to the porous support layer 120. In this case, desirably, the hydrogel is applied a plurality of times to the porous support layer 120.

According to design conditions, a small amount of general adhesion constituent may be applied to top of the porous support layer 120. This enables the functional layer 200 to be kept brought into contact with top of the porous support layer 120 in a process of shipping a product.

If a user applies a small amount of water to the water adhesion layer 100 made through the above-mentioned process and then attaches the water adhesion layer 100 to his or her nail, the water-soluble adhesion function of the water adhesion constituent 110 is exerted so that the patch is attached to the nail.

At the moment when the patch is attached to the nail, it is compressed so that the water adhesion constituent 110 passes through top of the porous support layer 120 and has an influence on the functional layer 200 and the beauty cover layer 300 formed thereon, thereby naturally allowing the patch to be firmly attached to the nail, which will be described later. As the patch is compressed, that is, an antibacterial constituent 210 and a nutrition constituent 230 of the functional layer 200 are naturally applied to the nail, and the porous support layer 120 of the water adhesion layer 100, a porous support layer 220 of the functional layer 200, and the beauty cover layer 300 form the structure of the patch, so that the structure of the patch becomes firm.

As time passes, further, the adhesion force decreases due to the consumption of the water adhesion constituent 110, and therefore, the patch is easily detachable from the nail through water.

The functional layer 200 serves to apply the antibacterial and nutrition constituents to the nail and includes the antibacterial constituent 210, a porous support layer 220, and the nutrition constituent 230. According to design conditions, the antibacterial constituent 210 and the nutrition constituent 230 may be changed to each other in position.

*

The antibacterial constituent 210 has an antibacterial function against fungus. Desirably, the antibacterial constituent 210 has an antibacterial function against Trichophyton rubrum and Trichophyton mentagrophytes. Otherwise, the antibacterial constituent 210 having the antibacterial function against the above-mentioned fungus is made of a natural extract extracted from a natural product. For example, the natural extract includes an amino-polysaccharide, chitosan, Cedrela sinensis extract, etc. According to design conditions, the antibacterial constituent 210 may have a preventive function so that it may selectively include a constituent having an antifungal function and a constituent having an antibacterial function weaker than the antifungal function.

The porous support layer 220 has the shape of a patch, while supporting the antibacterial constituent 210 and the nutrition constituent 230. The porous support layer 220 has the same design condition as the porous support layer 120 of the water adhesion layer 100 and adopts the material and shape of the porous support layer 120 of the water adhesion layer 100.

According to design conditions, the porous support layer 220 may be made of a material having holes smaller than ventilation holes of the porous support layer 120 of the water adhesion layer 100. This enables the discharge speed of the constituent formed on top of the porous support layer 220 in a downward direction to be adjusted. As the patch is compressed, that is, the functions of the lower layer constituents are first exerted, and next, the functions of the upper layer constituents are slowly exerted.

Further, an attached area of the porous support layer 120 is enlarged to ensure the adhesion to the beauty cover layer 300. This will be described later.

The nutrition constituent 230 serves to apply moisture and nutrition to the nail. For example, the nutrition constituent 230 includes lavender essential oil, citrus junos oil, lime oil, phytoncide essential oil, etc.

A method for manufacturing the functional layer 200 will be understood through the above-mentioned method for manufacturing the water adhesion layer 100.

That is, solution spraying and drying are repeatedly carried out a plurality of times to form the antibacterial constituent 210 and the nutrition constituent 230 on underside and top of the porous support layer 220. Otherwise, solution spraying, powder application, and powder drying are repeatedly carried out a plurality of times. Otherwise, either the antibacterial constituent 210 or the nutrition constituent 230 or both of them is (are) made of hydrogel and then applied onto the porous support layer 220.

The beauty cover layer 300 has a given pattern for beauty printed on top thereof and an adhesion constituent applied to underside thereof. The beauty cover layer 300 has the shape of the nail, while covering the surface of the nail. For example, the beauty cover layer 300 is made of a PET film.

According to design conditions, the beauty cover layer 300 may be formed by applying a general adhesion constituent to the underside of a film commonly used in a nail sticker field. Otherwise, the beauty cover layer 300 may be made of a material more rigid than the commonly used film.

According to design conditions, the application of the general adhesion constituent to the underside of the beauty cover layer 300 and the formation of the water adhesion layer 100 on the underside of the beauty cover layer 300 may be carried out together. Otherwise, only the water adhesion layer 100 may be formed on the underside of the beauty cover layer 300.

At the time when a product is shipped, it is enough that the water adhesion layer 100, the functional layer 200, and the beauty cover layer 300 are attached to one another only to some extent. Surface tension occurs between the porous support layer 120 and the lower layer constituent of the functional layer 200 due to room temperature and humidity so that they are slightly attached to each other, and the porous support layer 220 and the beauty cover layer 300 are attached to each other through the surface tension between the beauty cover layer 300 and the upper layer constituent of the porous support layer 230 due to room temperature and humidity and the adhesion constituent on the underside of the beauty cover layer 300.

So as to allow the product to be shipped in a state of being more rigid, a small amount of general adhesion constituent is applied to top of the porous support layer 120, and further, the ventilation holes of the porous support layer 220 become reduced in size to increase the adhesion area of the functional layer 200.

If the patch is compressed at the moment when it is attached to the nail, the water adhesion constituent 110 passes through top of the porous support layer 120, thereby additionally generating strong surface tension through the constituent existing between the porous support layer 120 and the porous support layer 220, and further, such strong surface tension occurs between the porous support layer 220 and the beauty cover layer 300.

### Embodiment 2. Multi-Functional Nail Repair Patch for an Antibacterial Function and a Beauty Function

A nail repair patch according to a second embodiment of the present invention includes a water adhesion layer 100, a functional layer 200, and a beauty cover layer 300 (See FIG. 2).

Differences of the second embodiment of the present invention from the first embodiment of the present invention are as follows:
A nutrition constituent 230 and an antibacterial constituent 210 are formed on the underside of a porous support layer 220, not on top and underside of the porous support layer 220. The antibacterial constituent 210 and the nutrition constituent 230 are changed to each other in order. The formation of each layer is the same as in the first embodiment of the present invention.

### Embodiment 3. Multi-Functional Nail Repair Patch for an Antibacterial Function and a Beauty Function

Differences of the third embodiment of the present invention from the second embodiment of the present invention are as follows: A nutrition constituent 230 and an antibacterial constituent 210 are agitated to each other, turn into a single liquid, and are applied to the underside of a porous support layer 220 (See FIG. 3).

The liquid is sprayed and dried, and otherwise, the liquid is sprayed, powderized, applied, and dried. Alternatively, the liquid is applied to the form of a hydrogel. Otherwise, the liquid is agitated, subjected to sonication pre-treatment, and thus formed as a liposome by means of addition of a liposomal constituent, and one of the above-mentioned three methods is adopted to form the liquid.

### INDUSTRIAL APPLICABILITY

The repair patches manufactured according to the first to third embodiments of the present invention satisfy a consumer's needs for antibacterial and beauty functions, improve conveniences of use through which they can be attached easily to the nail only with the use of a small amount of water, are replaced with new ones after one to two days to provide excellent sanitary states, and give a fun to the user because the patch having a first pattern is changed to the patch having a second pattern, so that they are applicable in industrial fields and optimized in the commercial values thereof.

1. A nail repair patch comprising:
a water adhesion layer (100) for providing an attachable function to a nail by means of water;
a functional layer (200) having an antibacterial constituent and a nutrition constituent; and
a beauty cover layer (300) having a given pattern for beauty printed on top thereof and an adhesion constituent applied to underside thereof,
wherein the water adhesion layer (100) comprises:
a water adhesion constituent (110) for providing a water-soluble adhesion function; and
a porous support layer (120) for supporting the water adhesion constituent (110).

2. The nail repair patch according to claim 1, wherein the water adhesion constituent (110) comprises starch or gelatin.

3. The nail repair patch according to claim 1, wherein the functional layer (200) comprises the antibacterial constituent (210) and the nutrition constituent (230) formed on top and underside of a porous support layer (220) or on the underside of the porous support layer (220).

4. The nail repair patch according to claim 3, wherein the antibacterial constituent (210) and the nutrition constituent (230) are made to the form of a single liquid.

5. The nail repair patch according to claim 1, wherein the antibacterial constituent (210) comprises a constituent having an antibacterial function against fungus.

6. The nail repair patch according to claim 1, wherein the nutrition constituent (230) comprises a constituent for the moisturization and nutrition of the nail.

7. The nail repair patch according to claim 1, wherein the adhesion constituent on the underside of the beauty cover layer (300) is the same as the water adhesion constituent (110).

8. The nail repair patch according to claim 3, wherein the porous support layer (220) has smaller ventilation holes than the porous support layer (120).

9. The nail repair patch according to claim 1, wherein the water adhesion constituent (110) is formed by a process in which a solution containing a water adhesion material is sprayed and dried, by a process in which after the solution has been sprayed, the powder of the solution is applied and dried, or by a process in which the water adhesion material is made into a hydrogel and the hydrogel is applied.

10. The nail repair patch according to claim 1, wherein the antibacterial constituent (210) and the nutrition constituent (230) are formed by a process in which an antibacterial material solution or a nutrition material solution is sprayed and dried, by a process in which after the solution has been sprayed, the powder of the solution is applied and dried, or by a process in which an antibacterial material or a nutrition material is made into a hydrogel and the hydrogel is applied.

11. The nail repair patch according to claim 2, wherein the functional layer (200) comprises the antibacterial constituent (210) and the nutrition constituent (230) formed on top and underside of a porous support layer (220) or on the underside of the porous support layer (220).

12. The nail repair patch according to claim 11, wherein the antibacterial constituent (210) and the nutrition constituent (230) are made to the form of a single liquid.

13. The nail repair patch according to claim 11, wherein the porous support layer (220) has smaller ventilation holes than the porous support layer (120).

14. The nail repair patch according to claim 2, wherein the antibacterial constituent (210) comprises a constituent having an antibacterial function against fungus.

15. The nail repair patch according to claim 2, wherein the nutrition constituent (230) comprises a constituent for the moisturization and nutrition of the nail.
